(19) United States
(12) Patent Application Publication
(10) Pub. No.: US 2013/0058926 A1
(43) Pub. Date: Mar. 7, 2013

(54) METHOD FOR ALLEVIATING AND/OR PREVENTING SKIN REDDENING

(71) Applicants: Kentaro Kajiya, Kanagawa (JP); Mika Sawane, Kanagawa (JP); Yuzo Yoshida, Kanagawa (JP); Katsujiro Sato, Tokyo (JP)

(72) Inventors: Kentaro Kajiya, Kanagawa (JP); Mika Sawane, Kanagawa (JP); Yuzo Yoshida, Kanagawa (JP); Katsujiro Sato, Tokyo (JP)

(73) Assignee: Shiseido Company, Ltd.

(21) Appl. No.: 13/597,526

(22) Filed: Aug. 29, 2012

Related U.S. Application Data
(60) Provisional application No. 61/528,907, filed on Aug. 30, 2011.

(51) Int. Cl. A61K 39/395; A61K 31/517; A61P 17/00; A61K 31/404; A61K 31/506; A61K 31/502; A61K 38/16; A61K 31/44

(52) U.S. Cl. 424/133.1; 514/8.1; 514/266.2; 514/346; 514/414; 514/275; 514/248

(57) ABSTRACT

A method of alleviating and/or preventing skin reddening for a subject in need thereof by means of inhibiting angiogenesis at the site of the reddened skin, is provided.

### FIELD OF THE INVENTION

The present invention relates to a method for alleviating and/or preventing skin reddening.

### BACKGROUND ART

Facial reddened skin has been frequently found in people with fair skin in European countries and the United States of America, but also found in Asians and African Americans. Skin reddening is thought to be a first step of Rosacea and is medically defined as transient or persistent facial erythema, visible blood vessels and telangiectasia¹.

Because reddened skin is found in cheeks, nose and central forehead, mainly in the center of face, reddened skin alters their social interactions, leading to problems on the job, in their marriage or in meeting new people^{2, 3}. In contrast our knowledge about the pathology of reddened skin is surprisingly scanty maybe because of the difficulty of getting biopsy samples of skin which often appear in the center of the face. Additionally, many studies especially for rosacea do not take into account the possible influence of cosmetic skin care regimens concomitant with topical treatments⁴. Therefore we try to determine the histology of reddened skin, which allow us to develop a possible method to prevent and/or improve the symptom of reddened skin.

Our previous studies have revealed that pronounced angiogenesis is induced by acute UVB irradiation of human and mouse skin^{5, 6}. Several angiogenesis factors, including vascular endothelial growth factor-A (VEGF-A), basic fibroblast growth factor and interleukin-8 have been found to be upregulated in UVB-irradiated skin⁷⁻⁹. Recently, we have shown that targeted overexpression of VEGF-A enhances sensitivity to UVB-induced cutaneous photodamage associated with pronounced angiogenesis, whereas a systemic blockade of VEGF-A attenuated vascular abnormality and erythema induced by UVB¹⁰, suggesting that a main angiogenesis factor in skin could be a keratinocyte-derived VEGF-A. However, any contribution of VEGF-A in the mechanism of facial reddened skin has been completely unclear not only for the limited histological research but for the lack of non-invasive quantification method for VEGF-A from skin.

### DISCLOSURE OF THE INVENTION

The aim of the present invention is to provide a novel method for alleviating and/or preventing skin reddening. As a result of extended studies, surprisingly, we found that angiogenesis, in particular, enhanced expression of VEGF-A, is involved in the cause of skin reddening. This means that inhibiting angiogenesis would result in alleviation and/or prevention of skin reddening.

Accordingly, the present invention provides a method of alleviating and/or preventing skin reddening for a subject in need thereof, by means of inhibiting angiogenesis at the site of the reddened skin. The method can be medical treatment or a cosmetic treatment.

Preferably, the inhibition of angiogenesis is attained by applying one or more vascular endothelial growth factor A (VEGF-A) inhibitor to said site.

The VEGF-A inhibitor can preferably be selected from the group consisting of Bevacizuma, Aflibercept (VEGF trap), Cediranib, Sorafenib, Sunitinib, Pazopanib and Vatalanib.

In another embodiment, the present invention provides a composition, preferably, a pharmaceutical or cosmetic composition, for alleviating and/or preventing skin reddening by means of inhibiting angiogenesis at the site of the reddened skin.

Preferably, the composition comprises one or more vascular endothelial growth factor A (VEGF-A) inhibitor which inhibits angiogenesis.

The VEGF-A inhibitor can be selected from the group consisting of Bevacizuma, Aflibercept (VEGF trap), Cediranib, Sorafenib, Sunitinib, Pazopanib and Vatalanib.

### BRIEF DESCRIPTIONS OF DRAWINGS

FIG. 1 shows comparison of the appearances of facial skin (A: non red skin; B: reddened skin). It also shows comparison of the non-reddened and reddened skins by use of high-magnification video microscope (VMS) (C: non red skin; D: reddened skin).

FIG. 2 shows comparison of the non-reddened and reddened skins by means of hematoxylin-eosin staining (A: non red skin; B: reddened skin). It also shows comparison of the non-reddened and reddened skins by means of immunofluorescence analysis using a proliferation marker Ki67 (C: non red skin; D: reddened skin). Also shown are comparison of the epidermal thickness and the ratio of Ki67 positive cells between non-reddened and reddened skins (E: non red skin; F: reddened skin).

FIG. 3 shows comparison of the non-reddened and reddened skins by means of immunofluorescence analysis using a blood vessel marker CD31 (A: non red skin; B: reddened skin). It also shows comparison of the non-reddened and reddened skins by superposing the immunofluorescence analysis result on the basis of CD31 with the immunofluorescence result on the basis of Ki67 (C: non red skin; D: reddened skin). Also shown are comparison of the epidermal thickness and the ratio of Ki67 positive cells between non-reddened and reddened skins (E: non red skin; F: reddened skin).

FIG. 4 shows comparison of the expressions of VEGF-A by means of immunochemistry staining between non-reddened skin (A) and reddened skin (B).

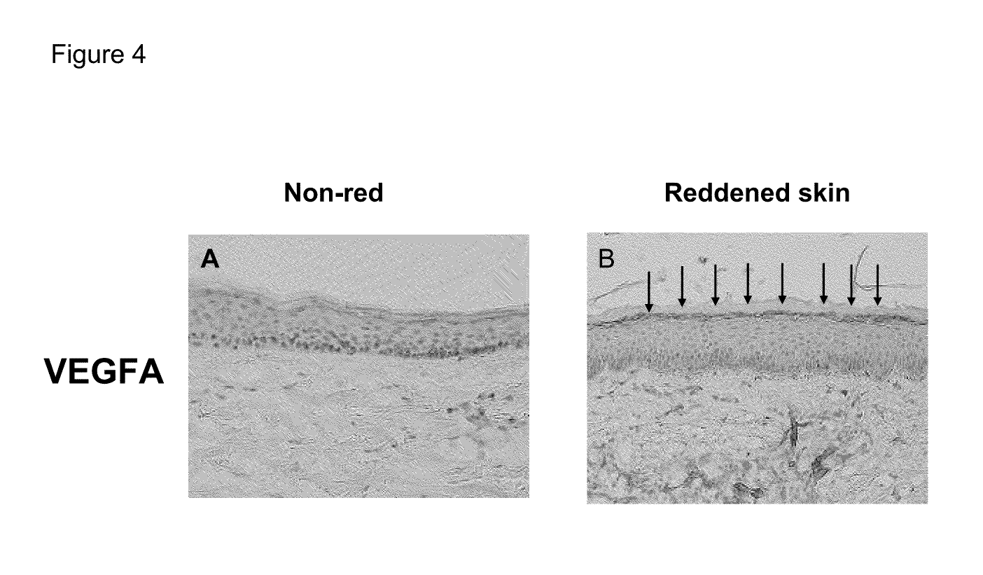

### BEST MODE FOR CARRYING OUT THE INVENTION

Inhibition of angiogenesis can be achieved by applying a drug having anti-angiogenesis activity to the site of skin, in particular, epidermis, of a subject in need of alleviation and/or prevention of skin reddening. The site of skin can be a portion which already shows reddening or a portion where prevention of reddening is desired. Thus, a composition, and particularly an external preparation for skin, containing such a drug as an active ingredient thereof is expected to demonstrate superior action which alleviates and/or prevents skin reddening, and is useful as a skin care pharmaceutical, over-the-counter drug or cosmetic.

Inhibition of angiogenesis in the epidermal cells can also be achieved by various genetic engineering technologies using, for example, RNA interference, anti-sense RNA-DNA, peptide and RNA-DNA aptamers, site-specific deletion, homologous recombination, dominant negative alleles or intrabodies.

Drug having anti-angiogenesis activity, in particular, VEGF-A inhibitory activity, can be any compounds already known to have such an activity or ones to be discovered in the future to have such an activity, and includes polypeptides or protein, such as monoclonal antibody, peptides, hormones, low molecular weight compound, or plant derived preparation such as crude drug or the like. Examples thereof are, but not limited to, Bevacizuma, Aflibercept (VEGF trap), Cediranib, Sorafenib, Sunitinib, Pazopanib and Vatalanib.

The drug having anti-angiogenesis activity is applied in the form of, for example, an aqueous solution, oily liquid, other type of solution, milky liquid, cream, gel, suspension, microcapsules, powder, granules, capsules or solid preparation. After having prepared the drug having anti-angiogenesis activity formed using known methods of the prior art, the drug can be coated, adhered, sprayed, injected or inserted into the body in the form of, for example, a lotion preparation, milky liquid preparation, cream preparation, ointment preparation, plaster preparation, poultice preparation, aerosol preparation, water-oil, bilayer preparation, water-oil-powder trilayer preparation or injection preparation. There are no particular limitations on the aforementioned extract in formulation to be used as inhibiting angiogenesis, and the amount thereof in terms of dry weight based on the total weight of the formulation can be, e.g., 0.000001 to 5% by weight, e.g., 0.00001 to 3% by weight or 0.00001 to 1% by weight.

Among these drug forms, externally applied skin preparations such as lotion preparations, milky lotion preparations, cream preparations, ointment preparations, plaster preparations, poultice preparations and aerosol preparations are contemplated as drug forms for the object of the present invention. Furthermore, externally applied skin preparations as referred to here include prescription pharmaceuticals, over-the-counter drugs and cosmetics.

The above-mentioned formulations suitably incorporates known vehicles and fragrances and the like corresponding to the desired drug form, as well as, for example, oils, surfactants, antiseptics, metal ion chelating agents, water-soluble polymers, thickeners,—pigments and other powdered components, ultraviolet protectants, moisturizers, antioxidants, pH adjusters, cleansing agents, drying agents or emulsions. Moreover, other pharmaceutically active ingredients can also be incorporated in the anti-graying agent of the present invention within a range that does not impair the desired effects thereof.

### EXAMPLES

### Material and Methods

### Imaging

The facial appearance was photographed and also imaged by using videomicroscope (VMS) (INT-200, Integral, Tokyo, Japan) as previously described¹¹. The field of view of the VMS was 585 by 410 μm (720 by 540 pixels). In VMS images, the features of capillary blood vessels were evaluated and color space (L*a*b*) of facial skin was also determined.

### Human Skin Samples

We obtained 2 mm biopsy from facial skin of eight female volunteers and proceed for histological analyses. All procedures involving human subjects were approved by the Institutional Review Board of Shiseido Research Center, and all subjects provided written informed consent.

### Immuno Stains

Immunofluorescence analysis was performed on 6-μm cryostat sections of skins, using mouse monoclonal antibodies against CD31 (BD Biosciences, San Diego, Calif.), against Ki-67 Antigen (DAKO cytomation, Glostrup, Denmark), and against VEGF-A (LAB VISION, Fremont, Calif.). Corresponding secondary antibodies labeled with AlexaFluor488 or AlexaFluor594 (Molecular Probes, Eugene, Oreg.) were used. Routine hematoxylin-eosin staining was also performed. Sections were examined with an Olympus AX80T microscope (Olympus, Tokyo, Japan) and images were captured with a DP controller digital camera (Olympus). Morphometric analyses were performed using IP-LAB software (Snanalytics, Fairfax, Va.) as described. Three different fields of each section were examined and the number of vessels per square micrometer, the average vessels size and the relative tissue area occupied by lymphatic vessels were determined in the dermis in an area within 200 μm distance from the epidermal-dermal junction. The unpaired Student t-test was used to analyze differences in microvessel density and size. Statistical analyses were performed using the unpaired student's t-test.

### Results

### Visible Blood Vessels Found in Reddened Skin

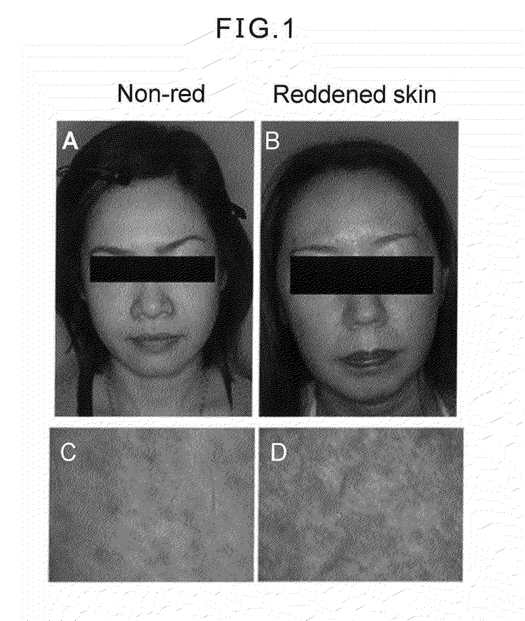

Reddened skin was found by red appearance of facial skin especially in cheeks and central forehead as compared to non-red skin (FIG. 1A, B). By using high-magnification videomicroscope (VMS), the skin color was defined as the color space (L*a*b*), which is also known as CIELAB. We next classified 8 healthy volunteers into 2 groups (reddened skin and non-red skin) according to their facial color space, b.

Dilation of capillary vessels were frequently found in the facial reddened skin as compared to non-red skin (FIG. 1C, D). The changes in VMS images were monitored by focusing on the image color of pigmentation as well as on the dilation of capillary blood vessels.

### Epidermal Hyperplasia and Inflammation in Reddened Skin

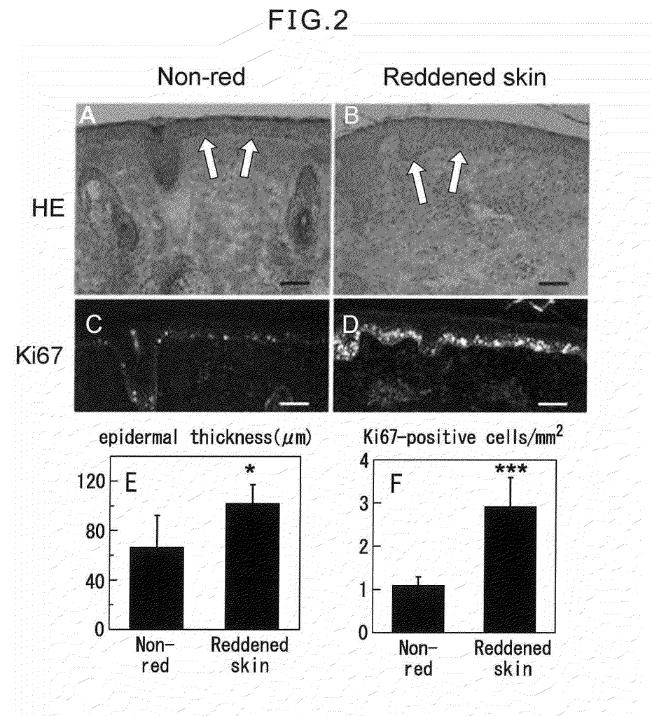

To determine the histological change in reddened skin, we obtained 2 mm of skin biopsy of reddened skin from 8 people. Hematoxylin-eosin staining of skin section revealed that reddened skin showed characteristics of epidermal hyperplasia as compared to non-red skin (FIG. 2A, B; see the portions indicated by arrows). Moreover, immunofluorescence analysis for a proliferation marker, Ki67 demonstrated that increased Ki67-positive cells were found in the epidermis of reddened skin as compared to non-red skin (FIG. 2C, D). Morphometric analysis confirmed that epidermal thickness and the number of Ki67-positive cells were markedly increased in reddened skin (FIG. 2E, F).

### Angiogenesis and Blood Vascular Enlargement in Reddened Skin

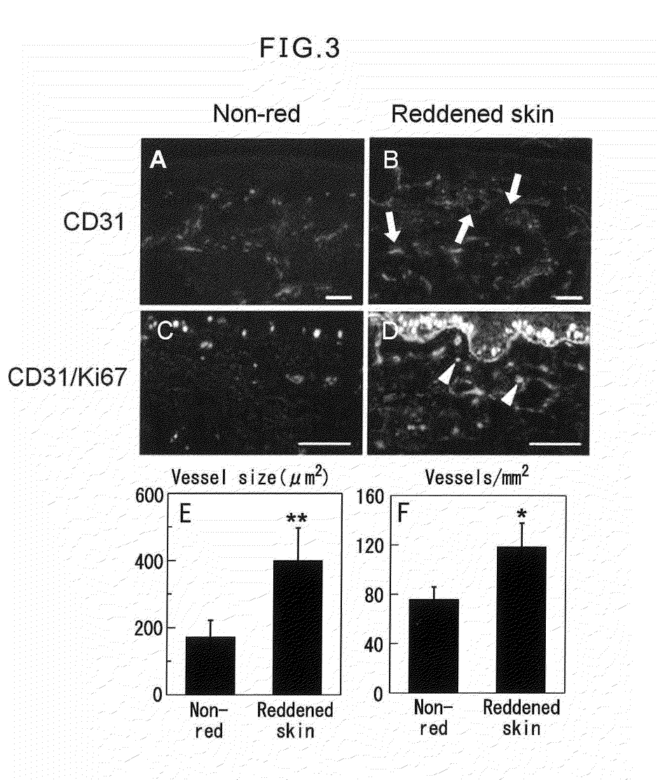

Since we found visible blood vessels by using video-microscope, we next performed, immunofluorescence analysis using antibodies against a blood vessel marker, CD31. As a result, we found that blood vessels were pronouncedly enlarged in the reddened skin (FIG. 3A, B see the portions indicated by arrows). Moreover, more blood vessels were found in the reticular dermis of reddened-skin as compared to non-red skin. Morphometric analysis showed the significant increase of size and density of blood vessels in the skin of reddened skin as compared to non-red skin (FIG. 3 E, F). Therefore we determined if the increase of blood vessels found in reddened skin was because of increased proliferation of blood vessels, we further performed the double immunofluorescence analysis using antibodies against CD31 and a proliferation marker, Ki67, revealing that the number of Ki67-positive CD31 vessels was increased in reddened skin, indicating that angiogenesis was found in the skin of reddened skin (FIG. 3C, D).

### REFERENCE

1. Wilkin J, Dahl M, Detmar M, et al. Standard grading system for rosacea: report of the National Rosacea Society Expert Committee on the classification and staging of rosacea. J Am Acad Dermatol. 2004;50:907-912.

2. Crawford GH, Pelle MT, James WD. Rosacea: I. Etiology, pathogenesis, and subtype classification. J Am Acad Dermatol. 2004;51:327-341; quiz 342-324.

3. Wilkin J, Dahl M, Detmar M, et al. Standard classification of rosacea: Report of the National Rosacea Society Expert Committee on the Classification and Staging of Rosacea. J Am Acad Dermatol. 2002;46:584-587.

4. Gallo R, Drago F, Paolino S, Parodi A. Rosacea treatments: What's new and what's on the horizon? Am J Clin Dermatol. 2010;11:299-303.

5. Yano K, Kadoya K, Kajiya K, Hong YK, Detmar M. Ultraviolet B irradiation of human skin induces an angiogenic switch that is mediated by upregulation of vascular endothelial growth factor and by downregulation of thrombospondin-1. Br J Dermatol. 2005;152:115-121.

6. Yano K, Oura H, Detmar M. Targeted overexpression of the angiogenesis inhibitor thrombospondin-1 in the epidermis of transgenic mice prevents ultraviolet-B-induced angiogenesis and cutaneous photo-damage. J Invest Dermatol. 2002;118:800-805.

7. Kramer M, Sachsenmaier C, Herrlich P, Rahmsdorf HJ. UV irradiation-induced interleukin-1 and basic fibroblast growth factor synthesis and release mediate part of the UV response. J Biol Chem. 1993;268:6734-6741.

8. Strickland I, Rhodes LE, Flanagan BF, Friedmann PS. TNF-alpha and IL-8 are upregulated in the epidermis of normal human skin after UVB exposure: correlation with neutrophil accumulation and E-selectin expression. J Invest Dermatol. 1997;108:763-768.

9. Bielenberg DR, Bucana CD, Sanchez R, Donawho CK, Kripke ML, Fidler IJ. Molecular regulation of UVB-induced cutaneous angiogenesis. J Invest Dermatol. 1998;111:864-872.

10. Hirakawa S, Fujii S, Kajiya K, Yano K, Detmar M. Vascular endothelial growth factor promotes sensitivity to ultraviolet B-induced cutaneous photodamage. Blood. 2005;105:2392-2399.

11. Yamashita T, Negishi K, Hariya T, Yanai M, Iikura T, Wakamatsu S. In vivo microscopic approaches for facial melanocytic lesions after quality-switched ruby laser therapy: time-sequential imaging of melanin and melanocytes of solar lentigo in Asian skin. Dermatol Surg. 2010;36:1138-1147.

What is claimed is:

1. A method of alleviating and/or preventing skin reddening for a subject in need thereof by means of inhibiting angiogenesis at the site of the reddened skin.

2. The method of claim 1 wherein inhibition of angiogenesis is attained by applying one or more vascular endothelial growth factor A (VEGF-A) inhibitor to said site.

3. The method of claim 2, wherein the VEGF-A inhibitor is selected from the group consisting of Bevacizuma, Aflibercept (VEGF trap), Cediranib, Sorafenib, Sunitinib, Pazopanib and Vatalanib.
